(19) United States
(12) Patent Application Publication
(10) Pub. No.: US 2020/0375283 A1
(43) Pub. Date: Dec. 3, 2020

(54) Wearable Therapy System

(71) Applicant: In-Covery Systems, Inc., Prescott, AZ (US)

(72) Inventors: James R. Check, Prescott, AZ (US); Heather S. Royer, Prescott, AZ (US)

(73) Assignee: In-Covery Systems, Inc., Prescott, AZ (US)

(21) Appl. No.: 16/428,237

(22) Filed: May 31, 2019

(51) Int. Cl. A41D 13/12; A61F 7/10; A41B 1/08; A41D 1/02; A41D 1/04; A41D 1/06; A41D 19/00; A41B 3/00

(52) U.S. Cl. CPC A41D 13/1236; A61F 7/10; A41B 1/08; A41D 1/02; A41D 1/04; A41B 2400/32; A41D 19/0037; A41B 3/00; A41D 2300/32; A41D 2400/32; A41D 1/06

(57) ABSTRACT

A wearable therapy system comprises a therapy pack, a therapy pack holder including a body and a first fastener system, the body being configured to removably receive the therapy pack, and a therapy garment configured to be worn by a user and comprising at least one attachment area including a second fastener system. The therapy pack holder is removably attachable to the attachment area in a plurality of positions with the first fastener system engaging the second fastener system to secure the therapy pack in relation to the therapy garment.

### BACKGROUND

### Field of the Disclosure

The present disclosure relates generally to a wearable therapy system. More specifically, the present disclosure relates to a wearable therapy system comprising a wearable garment and therapeutic hot and cold packs capable of being attached to and detached from the wearable garment.

### Related Art

Heat therapy and cold therapy are commonly used to alleviate pain, swelling, inflammation, and other conditions related to surgery, injury, exercise, and natural conditions. Heat therapy improves circulation and blood flow to a particular area. Specifically, increasing the temperature of an afflicted area can soothe discomfort, increase muscle flexibility, and heal damaged tissue. Cold therapy reduces blood flow to a particular area, which can reduce inflammation and swelling. Additionally, cold therapy reduces nerve activity, which can relieve pain.

To apply heat or cold therapy, therapeutic pads (e.g., ice/heat packs) are commonly used on the afflicted area. Generally, a person holds the ice/heat pack in a desired position against the body for a time period, or attaches the ice/heat pack to the body via adhesive tape or an elastic bandage wrap. Such methods are cumbersome, irritating, and can be difficult for a single person to employ. In addition, such methods are hard to apply when the person is injured and can become loose or undone during movement. In view of the foregoing, it is desirable to provide a wearable therapy system capable of providing hot and cold therapies with convenience and ease.

### SUMMARY

The present disclosure generally relates to a wearable therapy system for an individual. Specifically, the wearable therapy system comprises a therapy pack, a therapy pack holder including a body and a first fastener system, the body being configured to be worn by a user and to removably receive the therapy pack, and a therapy garment comprising at least one attachment area including a second fastener system. The therapy pack holder is removably attachable to the attachment area in a plurality of positions with the first fastener system engaging the second fastener system to secure the therapy pack in relation to the therapy garment.

### BRIEF DESCRIPTION OF THE DRAWINGS

The foregoing features of the disclosure will be apparent from the following Detailed Description, taken in connection with the accompanying drawings, in which:

FIG. 1 is an illustration showing a therapy system of the present disclosure including a therapy garment, a therapy pack, and a therapy pack holder;

FIG. 2 is an illustration showing the therapy pack of the therapy system of FIG. 1 in greater detail;

FIG. 3A is a front view of the therapy pack holder of the therapy system of FIG. 1 in greater detail;

FIG. 3B is a rear view of the therapy pack holder of the therapy system of FIG. 1 in greater detail;

FIG. 4A is a front view of the therapy garment of the therapy system of FIG. 1 in greater detail; and

FIG. 4B is a rear view of the therapy garment of the therapy system of FIG. 1 in greater detail.

### DETAILED DESCRIPTION

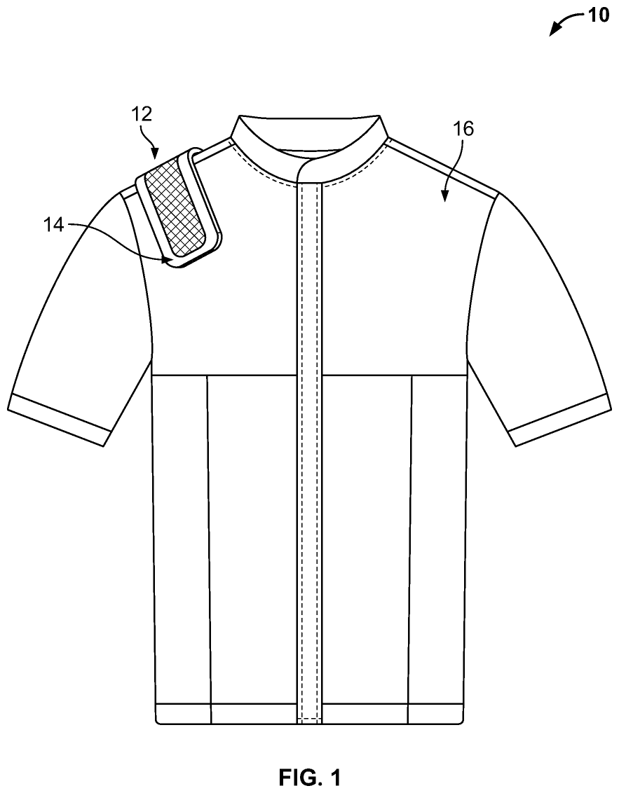

The present disclosure relates to a therapy system comprising a wearable garment and therapy pack capable of being attached to and detached from the wearable garment, as described in detail below in connection with FIGS. 1-4B.

FIG. 1 is an illustration showing the therapy system 10 of the present disclosure. The therapy system 10 comprises a therapy pack 12, a therapy pack holder 14, and a therapy garment 16. The therapy pack 12 can be, for example, a hot or cold pack/pad, and can be removably housed in the therapy pack holder 14. The therapy pack holder 14 can be attached to different locations on the therapy garment 16, and detached therefrom as desired. This allows the therapy pack 12 to provide therapy, e.g., hot or cold therapy, to a targeted location on a user. For example, the user (after a workout) can attach a cold therapy pack to their shoulder to alleviate pain, and a heated therapy pack to their chest to improve circulation and soothe discomfort.

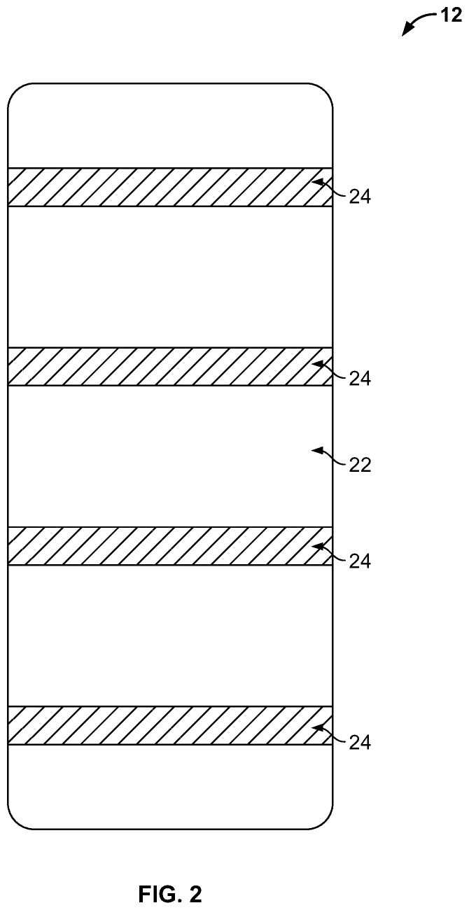

FIG. 2 is an illustration of the therapy pack 12. The therapy pack 12 comprises a flexible pouch 22 filled with a material capable of retaining and/or generating heat or cold, such as a hot pack or a cold pack, and one or more securing strips 24. The therapy pack 12 can be a reusable pack or a single use pack. The material inside the therapy pack 12 can comprise water, a refrigerant gel, any other liquid, ammonium nitrate, calcium ammonium nitrate, urea, hydroxyethyl cellulose, sodium polyacrylate, vinyl-coated silica gel, sodium acetate, iron powder, grains, or any other material/combination of materials capable of retaining or producing cold or heat.

The securing strips 24 enable the therapy pack 12 to be stably positioned within the therapy pack holder 14. The securing strips 24 can comprise any material with adhesive, fastening, or gripping properties, such as, but not limited to, silicone, a hook and loop fastener system (e.g., Velcro®), adhesive tape, rubber, etc. It should be understood that any amount of securing strips 24 can be attached to the pouch 22, and in any configuration.

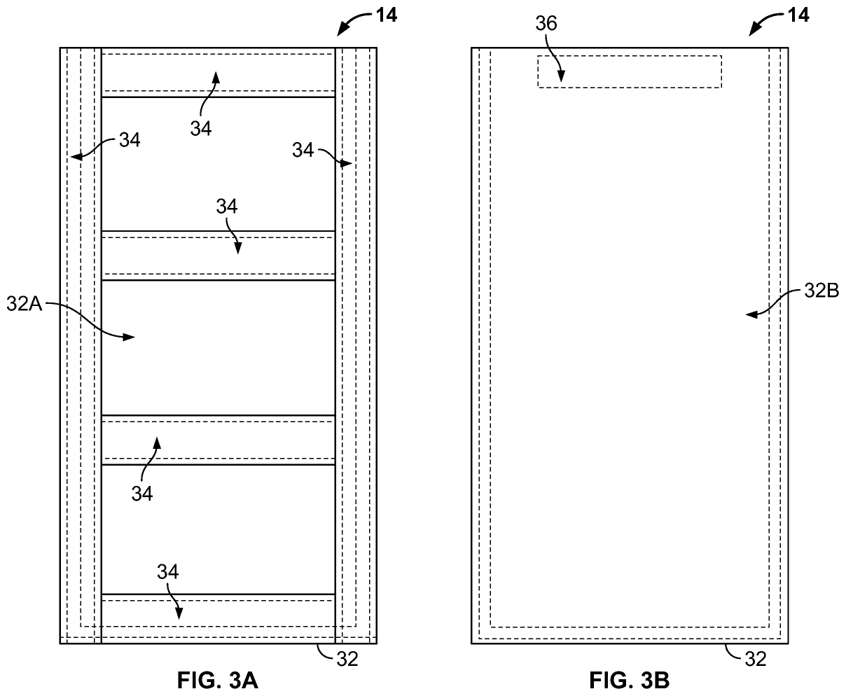

FIGS. 3A and 3B are first and second illustrations showing the therapy pack holder 14 of the therapy system 10. Specifically, FIG. 3A is a front view of the therapy pack holder 14 and FIG. 3B is a rear view of the therapy pack holder 14. The therapy pack holder 14 comprises a pouch 32 having a front side 32A and a back side 32A, a fastener system 34, and a closure mechanism 36. The pouch 32 can be formed of various materials, such as, a rubber material (e.g., neoprene, latex, other synthetic or natural rubbers), a cloth material, a synthetic material, a natural material, a material with dry wick capabilities, any other suitable material as known by those skilled in the art, or any combination of the foregoing materials. Additionally, the pouch 32 can be formed in various configurations, including but not limited to, a solid configuration, a mesh-type configuration, a netting-type configuration, a weave configuration, any other suitable combination as known by those skilled in the art, or any combination of the foregoing configurations. By way of example, the front side 32A of the pouch can comprise a first material in a solid configuration while the back side 32A of the pouch can comprise a second material in a mesh configuration.

The fastener system 34 is attached to the pouch 32 and can comprise any type of system capable of removably attaching the therapy pack holder 14 to the therapy garment 16. By way of example, the fastener system 34 discussed herein could be a hook and loop fastener system (or one component thereof), such as Velcro®. However, those skilled in the art would understand that any other suitable fastener system can be used for attaching and detaching the therapy pack holder 14 to the therapy garment 16, such as but not limited to, an adhesive system, a magnetic system, a zipper system, a button system, a clip system, etc. In another example, the therapy pack 12 itself can include a fastener system such that is can be attached directly to the therapy garment 16.

The hook and loop fastener system comprises a hook component having small hooks, and a loop component having small loops. Pressing the hook component to the loop component causes the hooks to attach to the loops and fasten the hook component to the loop component. By way of example, the fastener system 34 can comprise a hook component, and can attach to a loop component of the therapy garment 16 (or vice versa).

The pouch 32 can comprise one or more openings. The therapy pack 12 can be inserted into the pouch 32 via the one or more openings. The closure mechanism 36 is used to aid in securing the therapy pack 12 inside the pouch 32. For example, the closure mechanism 36 can comprise a hook and loop fastener system inside the pouch 32. Specifically, a hook component can be attached to an interior top edge of the back side 32B of the of the pouch and a loop component can be attached to an interior top edge of the front side 32A of the pouch. Alternatively, the closure mechanism 36 can comprise a flap system, an adhesive system, a zipper system, a button system, a magnetic system, a clip system, or any other suitable system to retain the therapy pack 12 inside the pouch 32.

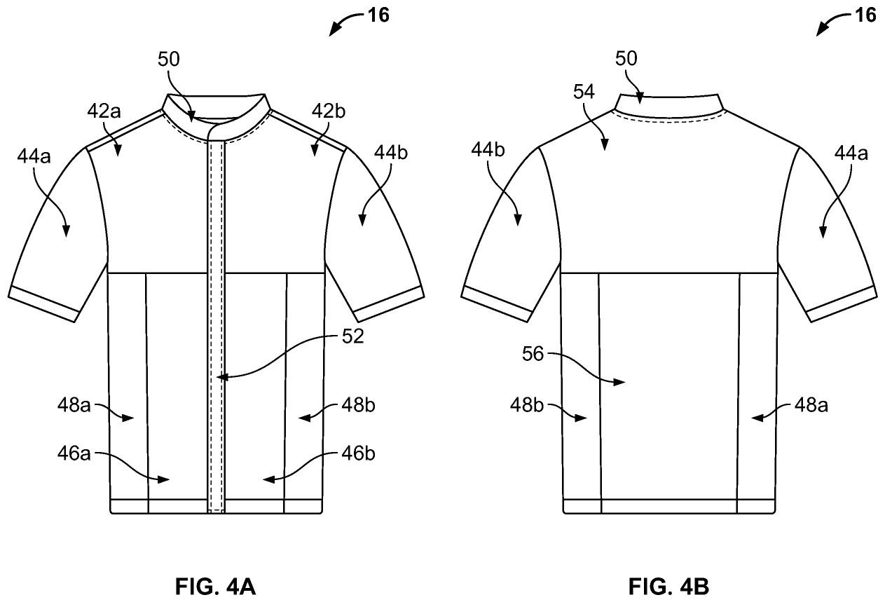

FIGS. 4A and 4B are illustrations showing the therapy garment 16 of the therapy system 10 in greater detail. Specifically, FIG. 4A is a front view of the therapy garment 16 and FIG. 4B is a back view of the therapy garment 16. The therapy garment 16 can comprise a plurality of sections, including a first front yoke 42a, a second front yoke 42b, a first sleeve 44a, a second sleeve 44b, a first midsection 46a, a second midsection 46b, a first side section 48a, a second side section 48b, a collar 50, a placket 52, a back yoke 54, and a back section 56. The entirety, or discrete portions of each section of the therapy garment 16 can comprise a fastener system 34 capable of having the therapy pack holder 14 removably attached thereto. By way of example, the first front yoke 42a, the second front yoke 42b, the first sleeve, 44a, the second sleeve 42b, and the back section 56 can comprise a loop component, to which a hook component of the therapy pack holder 14 can be attached. As such, the therapy pack holder 14 can be removably attached to any portion of the first front yoke 42a, the second front yoke 42b, the first sleeve, 44a, the second sleeve 42b, and the back section 56. However, it is understood that the garment sections can comprise any attachment system discussed herein. Further, it is noted that the garment section discussed is for illustrative purposes and that any combination of garment sections can be combined to form a single section or different sections.

Moreover, any combination of the foregoing sections can include a fastener system. It is noted that although entire sections are described as being formed of a fastener material, it should be understood that individual strips can be placed in various locations on one or more of the sections.

The collar 50 can be raised and comprise of a padded or soft material. This allows the collar 50 to lessen discomfort associated with the user using a sling (e.g., due to surgery, injury, etc.), placing the therapy pack holder 14 near the neck area, etc. The placket 52 can comprise an attachment system to close the therapy garment 16 onto the user. For example, the placket 52 can comprise a hook and loop fastener system, hook and loop buttons, a zipper system, a button system, etc.

As discussed above, the therapy system 10 allows the user to wear the therapy garment 16 and removably attach one or more therapy packs 12, either alone or held by a therapy holder 14, to the therapy garment 16. It is noted that the use of a shirt as the therapy garment 16 is by way of example only, and that other garments can be used as the therapy garment 16, including but not limited to, long sleeve shirts, compression garments, sweatshirts, hooded sweatshirts, jackets, pants, shorts, gloves, braces (e.g., shoulder braces, elbow braces, wrist braces, knee braces, ankle braces, etc.) hats, masks, etc.

Having thus described the system and method in detail, it is to be understood that the foregoing description is not intended to limit the spirit or scope thereof. It will be understood that the embodiments of the present disclosure described herein are merely exemplary and that a person skilled in the art may make any variations and modification without departing from the spirit and scope of the disclosure. All such variations and modifications, including those discussed above, are intended to be included within the scope of the disclosure.

1. A wearable therapy system, comprising,
a therapy pack;
a therapy pack holder including a body and a first fastener system, the body being configured to removably receive the therapy pack; and
a therapy garment configured to be worn by a user, the therapy garment comprising at least one attachment area including a second fastener system, wherein the therapy pack holder is removably attachable to the attachment area in a plurality of positions with the first fastener system engaging the second fastener system to secure the therapy pack in relation to the therapy garment.

2. The wearable therapy system of claim 1, wherein the therapy pack comprises a pouch filled with a material capable of retaining heat or cold, or generating heat or cold.

3. The wearable therapy system of claim 1, wherein the therapy pack comprises at least one securing strip.

4. The wearable therapy system of claim 3, wherein the securing strip comprises a silicone material or a rubber material.

5. The wearable therapy system of claim 3, wherein the therapy pack can be removably attached to the therapy garment with the at least one securing strip engaging the second fastener system.

6. The wearable therapy system of claim 1, wherein the first fastener system comprises a first component of a hook and loop fastener system, and the second fastener system comprises a second component of the hook and loop fastener system.

7. The wearable therapy system of claim 1, wherein the therapy pack holder is formed at least in part of a neoprene material

8. The wearable therapy system of claim 1, wherein the body of the therapy pack holder is formed at least in part from a mesh material.

9. The wearable therapy system of claim 1, wherein the therapy pack holder comprises a closure mechanism to secure the therapy pack with the therapy pack holder.

10. The wearable therapy system of claim 9, wherein the closure mechanism comprises a hook and loop fastener system.

11. The wearable therapy system of claim 1, wherein the therapy garment comprises a padded collar.

12. The wearable therapy system of claim 1, wherein the therapy garment comprises a placket.

13. The wearable therapy system of claim 12, wherein the placket comprises a third fastener system.

14. The wearable therapy system of claim 13, wherein the third fastener system of the placket comprises a hook and loop fastener system.

15. The wearable therapy system of claim 1, wherein the therapy garment is one of a shirt, a compression garment, a sweatshirt, a hooded sweatshirt, a jacket, a pair of pant, a pair of short, a glove, a shoulder brace, an elbow brace, a wrist brace, a knee brace, or an ankle brace.

16. The wearable therapy system of claim 1, wherein the second fastener system comprises a plurality of fastener strips.

17. The wearable therapy system of claim 1, wherein at least one of the first fastener system and the second fastener system comprises magnets.

18. The wearable therapy system of claim 1, wherein the therapy garment comprises a plurality of sections including one or more of a first front yoke, a second front yoke, a first sleeve, a second sleeve, a first midsection, a second midsection, a first side section, a second side section, a collar, a placket, a back yoke, and a back section.

19. The wearable therapy system of claim 18, wherein the at least one attachment area is provided at one or more of the plurality of sections.
